(19) United States
(12) Patent Application Publication
(10) Pub. No.: US 2002/0035352 A1
(43) Pub. Date: Mar. 21, 2002

(54) Absorbent article

(76) Inventors: Peter Ronnberg, Molndal (SE); Kent Vartiainen, Lerum (SE)

Correspondence Address: Ronald L. Grudziecki, Esq., BURNS, DOANE, SWECKER & MATHIS, L.L.P., P.O. Box 1404, Alexandria, VA, 22313-1404, US

(21) Appl. No.: 09/954,181

(22) Filed: Sep. 18, 2001

Related U.S. Application Data
(63) Continuation of application No. PCT/SE00/00445, filed on Mar. 7, 2000.

(30) Foreign Application Priority Data
Mar. 19, 1999 (SE) 9900998-7

(51) Int. Cl. A61F 13/15; A61F 13/20

(52) U.S. Cl. 604/366; 604/370; 604/372; 604/375; 604/378; 604/384

(57) ABSTRACT

Absorbent article such as a diaper, pant diaper, incontinence guard, sanitary napkin, wound dressing or the like comprising a layer of continuous fibers (9), so called tow, which is bonded in a random pattern where the bonding sites are randomly distributed in the xy- as well as in the z-direction of the layer, but where the fibers otherwise are substantially unbonded to each other. Preferably the layer is bonded by means of so called through-air-bonding. The layer can either be used as a liquid acquisition layer between the topsheet and the absorbent body, as a topsheet or as a combined topsheet and liquid acquisition layer.

### TECHNICAL FIELD

The present invention refers to an absorbent article such as a diaper, pant diaper, incontinence guard, sanitary napkin, wound dressing or the like of the kind comprising a liquid permeable topsheet, a liquid impervious backsheet and an absorbent body arranged therebetween.

### BACKGROUND OF THE INVENTION

Absorbent articles of the above mentioned kind are intended to absorb body liquids such as urine and blood. They usually comprise a liquid pervious topsheet, intended to be facing the wearer during use, in the form of a nonwoven material for example a spunbond material. It is also known to incorporate a liquid acquisition layer between the topsheet and the absorbent body, said liquid acquisition layer having the ability to quickly receive large amounts of liquid, to distribute it and temporarily store it before it is absorbed by the underlying absorbent body. This is important especially in today's thin compressed absorbent bodies often with a high amount of so called superabsorbents, which have a high absorption capacity but in many cases a too low absorption speed in order to immediately be able to absorb the large amount of liquid that can be discharged during a few seconds at urination. A porous relatively thick acquisition layer, for example in the form of a fibrous wadding, a carded fibrous web or other type of fibrous material has a high instantaneous liquid receiving capacity and can temporarily store liquid before it is absorbed by the absorbent body. The same applies for porous foam materials. The liquid is then drained successively to the underlying absorbent body, after which the acquisition layer again has capacity to receive liquid from a repeated wetting.

Examples of absorbent articles comprising such porous acquisition layer are for example disclosed in U.S. Pat. No. 3,371,667, EP-A-0,312,118 and EP-A-0,474,777.

The materials used today as acquisition layers in absorbent articles are mostly functioning well but are relatively expensive and can sometimes have an insufficient acquisition time, especially at the second and third wettings if large amounts of liquid are involved.

It is previously known through EP-A-0,391,814 and GB-B-2,209,672 to use continuous nonbonded synthetic fibers, so called tow, in absorbent articles to spread liquid in the longitudinal direction of the article.

Another problem is that conventional liquid pervious topsheet materials used for absorbent articles of this kind, usually a nonwoven material of synthetic fibers, e.g. a spunbond material, often has a lower acquisition rate for liquid than the acquisition layer, at which liquid can leak from the article before it reaches the acquisition layer. The problem can of course be solved by using a topsheet material which is very open and by that has a high liquid permeability. Such an open topsheet material can however cause problems with a too low strength and sharp fiber ends from the acquisition layer may penetrate the open topsheet material and irritate the user.

### OBJECTS AND MOST IMPORTANT FEATURES OF THE INVENTION

The object of the present invention is to provide a material having a high acquisition rate for liquid also at repeated wettings, has a high strength and wear resistance, high comfort and can be produced at a relatively low cost. This has according to the invention been provided by a layer of continuous fibers, so called tow, which is bonded in a random bonding pattern where the bonding sites are randomly distributed in the xy- as well as in the z-direction of the layer, but where the fibers otherwise are substantially unbonded to each other.

Bonding can be accomplished by so called through-air-bonding. At least a part of the continuous fibers can consist of a thermoplastic material, which softens or melts when heated. The hot air provides a bonding at the crossing sites of fibers that cross each other, at which a bonding of the layer is accomplished.

Alternatively particles of a thermoplastic material may be added to the layer, said material softens of melts i connection with the hot air blow-through and thus bonds the material together.

The material layer can be used as a liquid acquisition layer under a topsheet material, as a topsheet material or as an integrated topsheet/liquid acquisition layer.

### DESCRIPTION OF THE DRAWINGS

The invention will below be closer described with reference to some of the embodiments shown in the accompanying drawings.

FIG. 1 is a plan view of an absorbent article in the form of an incontinence guard.

FIG. 2 is a section according to the line II-II in FIG. 1.

FIG. 3 is a plan view of an absorbent article in the form of a diaper.

FIG. 4 shows schematically a piece of a fibrous material layer according to the invention.

FIGS. 5-8 are schematic exploded longitudinal sectional views of four different embodiments of the article according to the invention.

### DESCRIPTION OF EMBODIMENTS

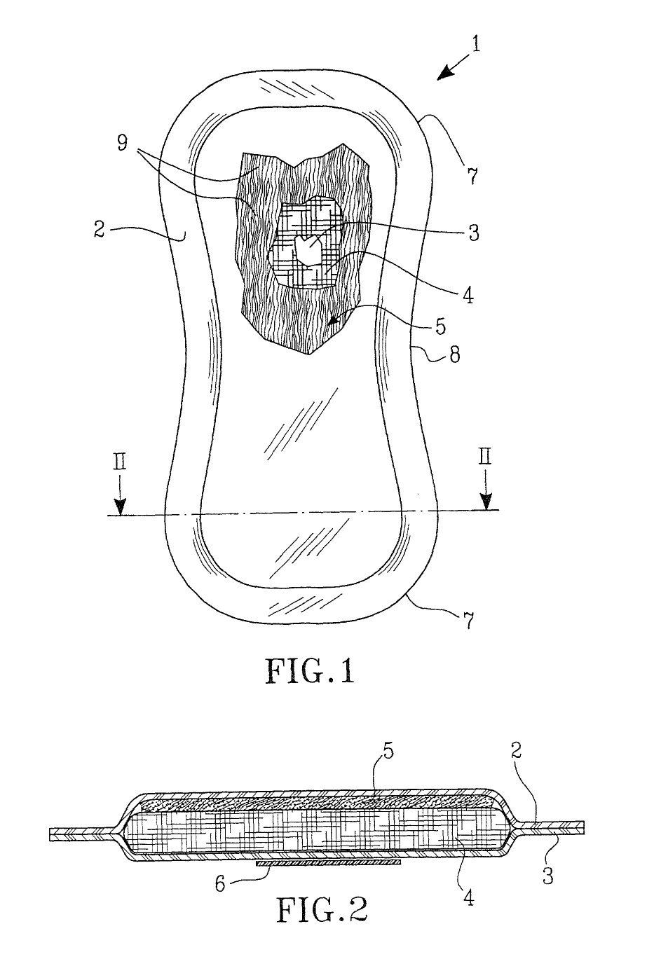

FIG. 1 and 2 show schematically an example of an incontinence guard 1 comprising a liquid pervious topsheet 2, a liquid impervious backsheet 3 and an absorbent body 4 enclosed therebetween. A porous resilient liquid acquisition layer 5 is arranged between the liquid pervious topsheet 2 and the absorbent body 4.

The liquid pervious topsheet 2 can comprise a nonwoven material, for example a spunbond material of synthetic filaments, a meltblown material, a thermobonded material or a bonded carded fibrous material. The liquid impervious backsheet 3 can consist of a plastic film, a nonwoven material which is coated with a liquid impervious material or a hydrophobic nonwoven material which resists liquid penetration.

The topsheet 2 and the backsheet 3 have a larger surface area than the absorbent body 4 and the liquid acquisition layer 3 and extend outside the edges thereof. The layers 2 and 3 are interconnected within the projecting portions, for example by gluing or welding with heat or ultrasonic.

The absorbent body 4 can be of any conventional kind. Examples of common absorption materials are cellulosic fluff pulp, tissue layers, highly absorbent polymers (so called superabsorbents), absorbent foam materials, absorbent nonwoven materials and the like. It is common to combine cellulosic fluff pulp with superabsorbents in an absorbent body. It is also common to have absorbent bodies comprising layers of different materials with different properties concerning liquid acquisition capacity, liquid distribution capacity and liquid storage capacity. This is wellknown for the person skilled in the art and need not be described in detail. The thin absorbent bodies which are common in for example baby diapers and incontinence guards often consist of a compressed mixed or layered structure of cellulosic fluff pulp and superabsorbent.

On the outside of the liquid impervious backsheet 3 fastening means in the form of strips 6 of a selfadhesive glue are arranged. The glue areas 6 are preferably before use covered with a releasable protection strip (not shown) of a paper or plastic material treated with a release agent. In the embodiment shown the fastening means are in the form of longitudinal glue areas. A number of other types of glue patterns, for example transverse, are of course possible as well as other types of fastening means such as hook and loop, snap fasteners, girdles, special underpants or the like.

An incontinence guard of the kind disclosed in FIG. 1 is mainly intended to be used by persons suffering from relatively light incontinence and can easily be worn in a pair of ordinary underpants. The fastening means 6 serves to keep the incontinence guard in place during use.

The incontinence guard is hour glass shaped with broader end portions 7 and a more narrow crotch portion 8 located between the end portions. The crotch portion 8 is the portion of the incontinence guard that is intended during use to be worn in the crotch between the legs of the wearer and serve as a receiving portion for the discharged body fluid.

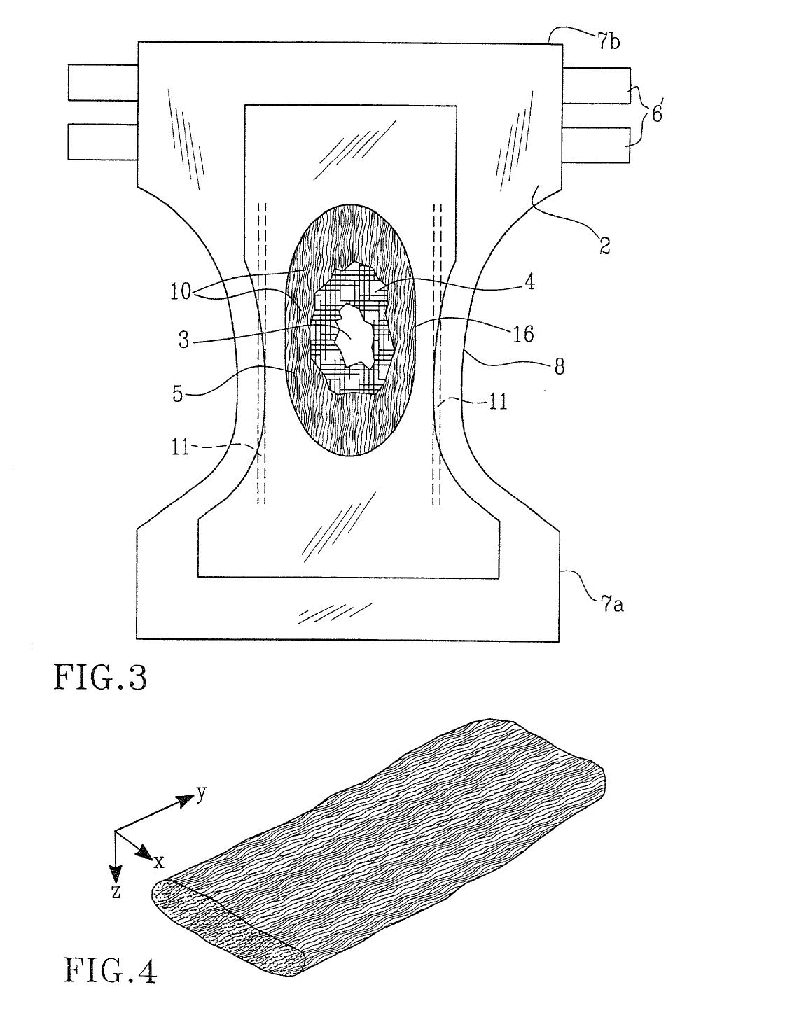

In FIG. 3 there is shown an absorbent article in the form of a diaper which like the above described incontinence guard comprises a liquid pervious topsheet 2, a liquid impervious backsheet 3 and an absorbent body 4 enclosed therebetween and further an acquisition layer 5 applied between the topsheet 2 and the absorbent body 4. In the embodiment shown the topsheet is provided with a hole 16 opposite the intended wetting area, at which the acquisition layer 5 is exposed directly towards the user in this area. Instead of one hole 16 several smaller holes may be arranged.

The diaper is intended to enclose the lower part of the user's trunk as a pair of absorbent pants. It has a front portion 7a intended during use to be worn against the front part of the user's body, a back portion 7b intended during use to be worn against the back part of the user, and a therebetween a narrower crotch portion 8 intended to be worn in the crotch part between the legs of the user. In order to fasten the diaper together to the desired pant shape tape strips 6′ around the waist of the wearer. Other fastening means such as hook and loop means (Velcro), hooks etc. are of course possible.

It should be noted that the incontinence guard and the diaper shown in the drawings and described above only are non-limiting examples of an absorbent article. Thus the shape of the article as well as the construction thereof can be varied. The absorbent article can also be a diaper, a pant diaper, a sanitary napkin or the like. The absorbent article can be disposable or reuseable. For reuseable articles other materials than the above described are however used as a liquid pervious topsheet and absorbent body respectively.

Between the liquid pervious topsheet 2 and the absorbent body 4 there is arranged a porous and resilient acquisition layer 5 having the ability to quickly receive large amounts of liquid and distribute the liquid and store it temporarily before it is absorbed by the underlying absorbent body 4. This ability should be essentially maintained also after wetting of the material. The acquisition layer 5 can either cover the entire absorbent body 4, extend outside thereof or cover only part of the central portions of the absorbent body.

According to the invention the acquisition layer 5 consists of a layer of continuous fibers 9, so called tow, which have been bonded together in a random bonding pattern where the bonding sites are randomly distributed in the xy- as well as in the z-direction of the layer, but otherwise are substantially unbonded to each other. The bonding is made in such a way that the layer has not been compressed at the bonding sites to any substantial degree. By this the bulk and high porosity of the material has been maintained, which promotes the liquid acquisition rate.

Bonding can be performed by so called through-air-bonding. At least a part of the continuous fibers shall consist of a thermoplastic material, which softens or melts by heat. Examples of thermoplastic polymers are polyolefines, polyamides, polyesters and the like. Also so called bicomponent fibers with a shell of a polymeric material having a lower melting point than the material in the core material of the fiber are included. The hot air provides that fibers crossing each other are bonded together at the crossing points, at which the layer is bonded.

Alternatively particles of a thermoplastic material can be added to the layer, said material softens or melts in connection with the through-air-bonding and thus provides that the continuous fibers are bonded together at the sites where they are in contact with the thermoplastic particles. The fibers may in this case be of any material.

Fiber tow is supplied in sacks or in the form of bales or rolls of continuous fibers, which either are straight, crimped or curled. Crimped or curled fibers are preferred in this case since they provide a very open and airy structure. The bales or the like are opened in special converting devices in which the fibers are separated from each other, stretched and spread out to an essentially evenly thick layer. The layer is bonded in the desired bonding pattern according to above and is cut in suitable lengths either before or after application in an absorbent article. The bonding can alternatively be made after cutting. A tow is a relatively cheap delivery form of fibers as compared to nonwoven, waddings or the like which are normally used as acquisition materials.

The fibers in the tow can be of any suitable material such as polyethylene, polypropylene, polyamide, polyester, polyvinyl acetate, cellulose acetate, regenerated cellulose such as viscose and rayon, or of bicomponent type with a shell of a polymer having a lower melting point and a core of a polymer having a higher melting point. Specially preferred are such fibers having a high resiliency, for example polyester or copolyester.

The fiber thickness can vary but should be in the interval 0.5 to 50 dtex, preferably 1.5 to 25 and most preferably 2 to 15 dtex, if the material is to be used as an acquisition layer. The open airy structure in combination with the relatively coarse fiber dimension gives a very rapid liquid acquisition. Besides the material is strong due to the continuous fibers which provide strength in the longitudinal direction, and the bonding pattern which provides strength in the transverse direction.

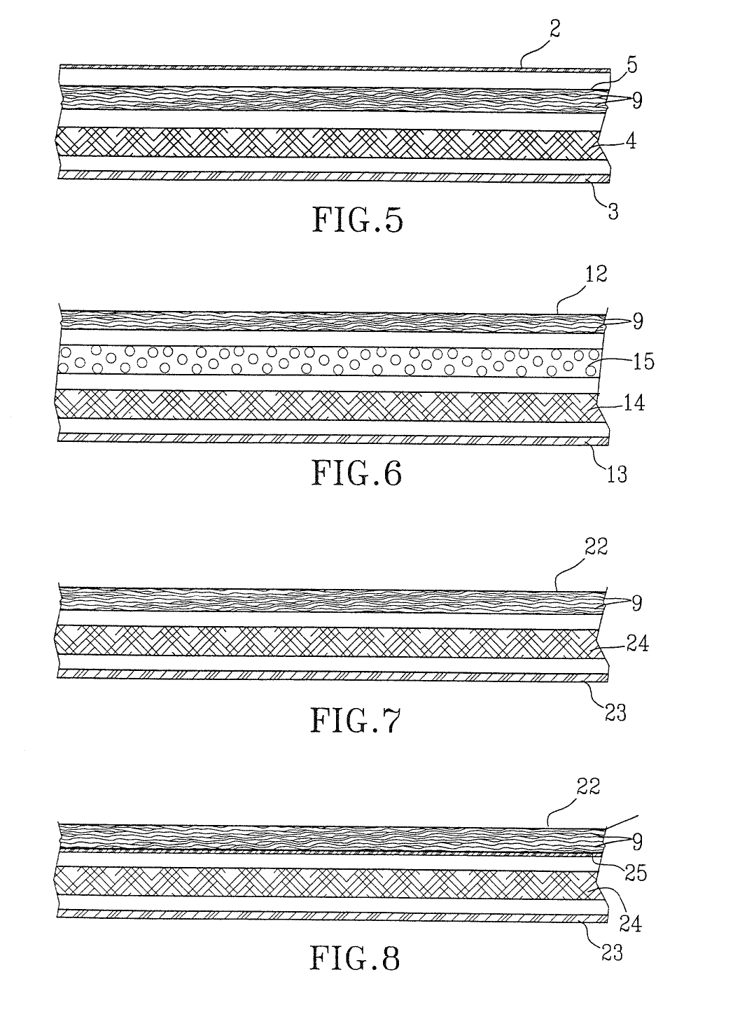

In the above example the material has been used as an acquistion layer 5 under a liquid pervious topsheet 2. This is also shown in FIG. 5. The basis weight of the bonded fiber tow should in this case be at least 10 g/m², preferably in the interval 10-1000 g/m², more preferably 30-700 g/m² and most preferably 30-350 g/m². The topsheet 2 can be of any optional kind, but preferably has a relatively open structure which permits a 2 5 quick liquid acquisition. The topsheet 2 may be bonded to the acquisition layer 5 in an appropriate way, e.g. glued.

In FIG. 6 there is shown an alternative embodiment in which the bonded fiber tow according to the invention is used as a liquid pervious topsheet 12. The basis weight should in this case be at least 5 g/m², preferably in the interval 5-500 g/m² and more preferably 5-200 g/m² and the fiber thickness should be in the interval 0.5-50 dtex, preferably 1.5-25 and more preferably 2-15 dtex. In other respect the material can be the same as described above. Under the topsheet 12 there is arranged an acquisition layer 15 which may be of optional kind. The absorbent article according to FIG. 6 further comprises an absorbent body 14 and a liquid pervious backsheet 13.

In the embodiment according to FIG. 7 the bonded fiber tow according to the invention has been used as a combined topsheet and acqusition material 22. The surface weight should in this case be at least 10 g/m², preferably in the interval 10-1000 g/m², more preferably 30-700 g/m² and most preferably 30-350 g/m² and the fiber thickness should be in the interval 0.5-50 dtex, preferably 1.5-25 and more preferably 2-15 dtex. The absorbent article according to FIG. 8 further comprises as usual an absorbent body 24 and a liquid pervious backsheet 23.

The embodiment according to FIG. 8 differs from the one disclosed in FIG. 7 by the fact that a support material 25 e.g. in the form of a nonwoven has been integrated on the underside of the combined topsheet/acquisition layer 22. Such a support material can of course alternatively or also be integrated to the upper side of the topsheet/acquisition layer 22 or to the acquisition layer 5 according to FIG. 5 or the topsheet 12 according to FIG. 6.

In the embodiments according to FIGS. 6-8 the bonded fiber tow according to the invention will be directly contacting the skin of the wearer. In this case there are high demands on the softness and comfort of the material. Since the material consists of continuous fibers there are no protruding sharp fiber ends which may irritate the skin, but the material is very soft and pliable. Besides it has a sufficient strength and wear resistance due to the longitudinal continuous fibers 9, which have been bonded together.

As was stated above it is preferred that crimped or curled fibers are used in the tow, since they provide an extra open and lofty structure. It is also possible to use a combination of straight and crimped or curled fibers.

It is also possible to use different kinds of fibers or different thicknesses of fibers in different parts of the tow, i.e. in different layers or zones thereof. By this it would be possible to create desired absorption patterns. It would also be possible to create gradients of different hydrophilicity and/or pore size. Superabsorbent fibers could also be admixed into the tow.

1. Absorbent article such as a diaper, pant diaper, incontinence guard, sanitary napkin, wound dressing, comprising a liquid permeable topsheet (2;12;22), a liquid impervious backsheet (3;13;23) and an absorbent body (4;14;24) arranged therebetween, the article comprising a layer (5) of continuous fibers (9), so called tow fibers, characterized in that the layer (5) of tow fibers (9) is bonded by through-air-bonding in a random bonding pattern where the bonding sites are randomly distributed in the xy- as well as in the z-direction of the layer, but where the fibers otherwise are unbonded to each other.

2. Absorbent article as claimed in claim 1, characterized in that at least a part of the tow fibers consist of a thermoplastic material.

3. Absorbent article as claimed in claim 1, characterized in that particles of a thermoplastic material have been added to the layer (5) of tow fibers (9) and that the layer is bonded by so called through-air-bonding.

4. Absorbent article as claimed in claim 1, characterized in that the layer of tow fibers (9) is used as a liquid acquisition layer (5) applied between the topsheet (2) and the absorbent body (3).

5. Absorbent article as claimed in claim 4, characterized in that the layer of tow fibers (9) has a basis weight of at least 10 g/m², preferably in the interval 10-1000 g/m², more preferably 30-700 g/m² and most preferably 30-350 g/m².

6. Absorbent article as claimed in claim 4, characterized in that the topsheet (2) in the intended wetting area is provided with at least one opening (16) through which the liquid acquisition layer (5) is exposed towards the user.

7. Absorbent article as claimed in claim 1, characterized in that the layer of tow fibers (9) is used as a liquid pervious topsheet (12).

8. Absorbent article as claimed in claim 7, characterized in that the layer of tow fibers (9) has a basis weight of at least 5 g/m², preferably in the interval 5-500 g/m², more preferably 5-200 g/m².

9. Absorbent article as claimed in claim 1, characterized in that the layer of tow fibers (9) is used as an integrated topsheet/liquid acquisition layer (22).

10. Absorbent article as claimed in claim 9, characterized in that the layer of tow fibers (9) has a basis weight of at least 10 g/m², preferably in the interval 10-1000 g/m², more preferably 30-700 g/m² and most preferably 30-350 g/m².

11. Absorbent article as claimed in claim 9, characterized in that at least a part of the tow fibers (9) in said layer are crimped or curled.

12. Absorbent article as claimed in claim 9, characterized in that the layer of tow fibers (9) is supported by a support layer (25), e.g. a nonwoven.

13. Absorbent article as claimed in claim 9, characterized in that the layer of tow fibers (9) comprises fibers of different material and/or different thicknesses.
